(19) United States
(12) Patent Application Publication
(10) Pub. No.: US 2007/0282446 A1
(43) Pub. Date: Dec. 6, 2007

(54) Body augmenter capable of being inserted with medicine

(76) Inventor: Kung-Chia Li, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/714,791

(22) Filed: Mar. 7, 2007

(30) Foreign Application Priority Data
May 30, 2006 (TW) 095209329

(51) Int. Cl. A61F 2/44

(52) U.S. Cl. 623/17.12

(57) ABSTRACT

A body augmenter capable of being inserted with medicine includes a nearly rectangular body augmenter to be planted in a vertebra, and the body augmenter has a fitst end and a second end and a chamber in its interior. The cross-sectional area of the first end gradually enlarges to the second end, and the chamber can be stored with medicine that can be changed in its kind, quantity and shape. An opening is provided in an outer surface of the body augmenter, communicating with the chamber so medicine may be inserted therethrough into the chamber. An opening sealer is further provided to close up the opening to hamper the medicine stored therein from falling out. Plural holes communicating with the chamber are provided in the outer surface of the body augmenter to enable the medicine therein to seep therethrough into a humane body.

### BACKGROUND OF THE INVENTION

1. Field of the Invention

This invention relates to a body augmenter capable of being inserted with medicine, particularly to one inserted therein with medicine, which can gradually seep into a humane body for treatment or sterilization after the body augmenter is planted in a vertebra.

2. Description of the Prior Art

Osteoporosis is caused by loss of calcium from the skeletal system of a human body, with the bones softening, loosening and becoming fragile, with the supporting force gradually decreasing, so fracture of the bone, especially such as vertebral body fractures including acute burst fracture or chronic compression fracture, may liable to occur. However, osteoporosis may differ according to races, living habits, heredity, medicine, sexual gland, function and gender.

If a vertebra is fractured owing to osteoporosis, especially by exterior force or disease, the vertebra may be damaged and dented. Then the surgical instrumentation may use two kinds of operation, to plant a posterior steel nail in the vertebra or an anterior bone graft in the vertebra. However, steel nail planting in the damaged vertebra and those on and under that damaged one at the same time may worsen the flexibility in the activity of the patient. On the other hand, the bone graft operation may involve a large cut scar in addition to a long period of time needed in the operation and the problem of bacterial infection. In view of the defects of the two means, there are three US patents of No. 2004/0215189 A1 titled “Pedicle augmenter for vertebral body reconstruction”. No. 2006/0085007 A1 titled “Distractable body augmenter capable of being planed through a pedicle for vertebral body reconstruction” and No. 2006/0025860 titled “Body augmenter capable of being plated through a pedicle for vertebral body reconstruction”, respectively offering a body augmenter to be planted in a vertebral body.

### SUMAMRY OF THE INVENTION

This invention has been devised to offer a body augmenter capable of being inserted with medicine, for producing function of treatment and fighting virus, bacteria, fungus etc. infectious sources or cancer cells of certain malignant tumors after it is planted in a vertebra or sterilization.

The feature of the invention is a body augmenter of a nearly rectangular shape to be planted in a vertebra, and the body augmenter has a first end and a second end, with the cross-sectional area of the first end gradually enlarging to the second end. The body augmenter is provided with a chamber wherein a medicine can be stored, and an opening is provided in an outer surface to communicate with the chamber so that the medicine can be inserted therethrough into the chamber. The body augmenter has its outer surfaces provided with plural holes commincating to the chamber so that the medicine stored therein may seep therethrough into a human body for treatment and fighting virus and infected cells or sterilization.

### BRIEF DESCRIPTION OF DRAWINGS

This invention will be better understood by referring to the accompanying drawings, wherein:

FIG. 1 is a perspective view of a first preferred embodiment of a body augmenter capable of being inserted with medicine in the present invention;

FIG. 2 is a cross-sectional view of the first preferred embodiment of a body augmenter capable of being inserted with medicine in the present invention;

FIG. 3 is a front cross-sectional view of the first preferred embodiment of a body augmenter capable of being inserted with medicine in the present invention;

FIG. 4 is a perspective view of a second preferred embodiment of a body augmenter capable of being inserted with medicine in the present invention;

FIG. 5 is a perspective view of a third preferred embodiment of a body augmenter capable of being inserted with medicine in the present invention; and,

FIG. 6 is a front cross-sectional view of the third preferred embodiment of a body augmenter capable of being inserted medicine in the present invention.

### DETAILED DESCRIPTION OF THE PREFERRED EMBODIMENTS

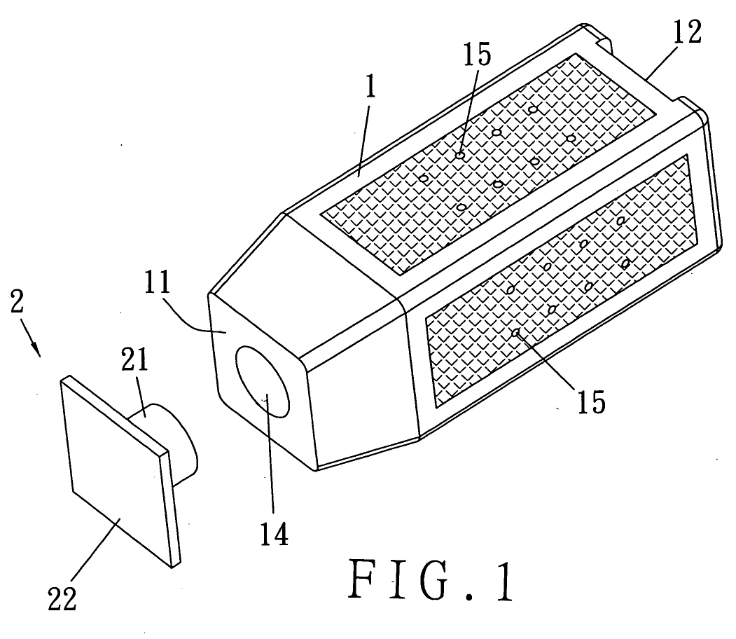

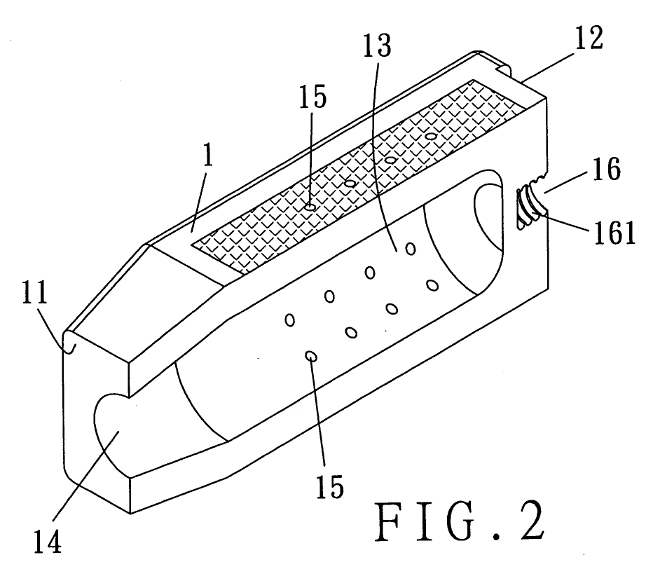

A first preferred embodiment of a body augmenter capable of being inserted with medicine in the present invention, as shown in FIGS. 1, 2 and 3, includes a body augmenter 1 provided with a first end 11 and a second end 12, and the cross-sectional area of the first end 11 gradually enlarges to the second end 12, in other words, the first end 11 has the smallest cross-sectional area and the second end 12 has the largest cross-sectional one. The body augmenter 1 has its outer surface marked with grains or vary small projections for upgrading friction, and a chamber 13 formed in its interior for storing medicine therein, and further the chamber 13 extends to a front outer surface to form an opening 14 so that medicine such as powder, greasy, liquid, gelatinous one, etc. may be inserted and filled therein through the opening 14. The medicine to be used can be changed in its sort, quantity, and shape according to practical necessity. In addition, plural holes 15 are provided in the outer surface, extending to the chamber 13 for the medicine stored in the chamber 13 may seep therethrough into a human body.

The medicine stored in the body augmenter 1 can be for treatment or sterilization, and it is placed in the chamber 13 thereof before the body augmenter 1 is planted in a vertebra, and then the cancer medicine may slowly seep and pervade into the body for containing spread of the infected cancer cells, or antibiotics may be stored in the chamber 13 for containing and fighting virus and lowering infection of bacteria after operation.

In addition, an opening sealer 2 of a T-shape is provided in the first embodiment for sealing the opening 14 after a medicine is stored in the chamber 13, having the size and shape corresponding to that of the opening 14, consisting of a block 21 fitting tightly in the opening 14 and a plate body 22 fixed with the block 21 for closely covering the surrounding surface of the opening 14 to prevent the medicine from falling out of the opening 14.

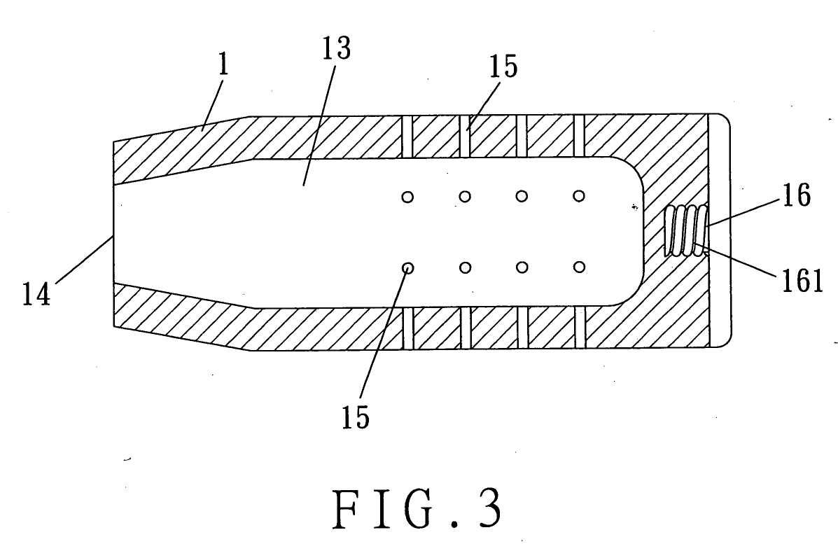

Next, the second end 12 of the body augmenter 1 is provided additionally with a tool hole 16, which can be used for stabilizing a tool of hole expanding, of clamping, etc, In the FIG. 3 illustrates a tool hole 16 with female threads 161 for engaging with threads of a tool. Anyway, the tool hole 16 can be of any shape according to practical need.

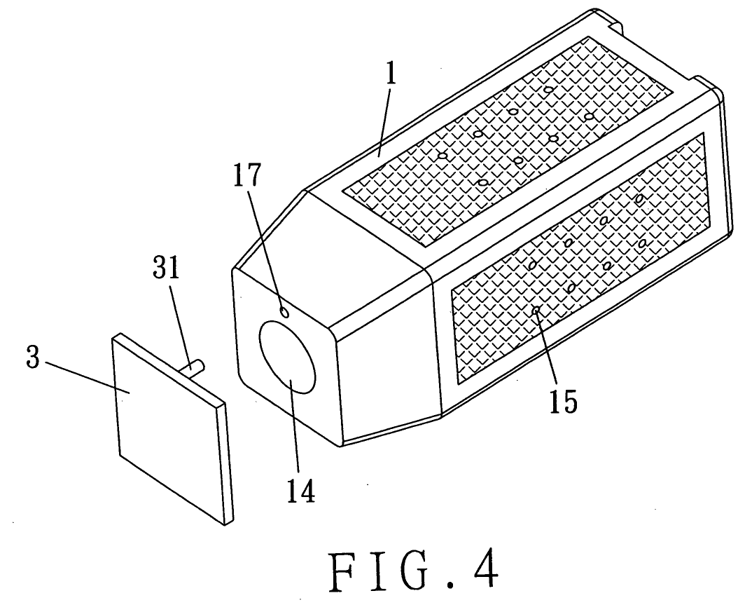

FIG. 4 shows a second preferred embodiment of a body augmenter capable of being inserted with medicine in the invention, having different structures from the first one that an opening sealer 3 is shaped as a plate, and pivotally connected to the body augmenter 1, having a pivot 31 fixed vertically with an upper inner surface of the plate body 3 to fit in a pivotal hole 17 bored in a front side surface of the body augmenter 1, enabling the opening sealer 3 to swing for closing and opening the opening 14.

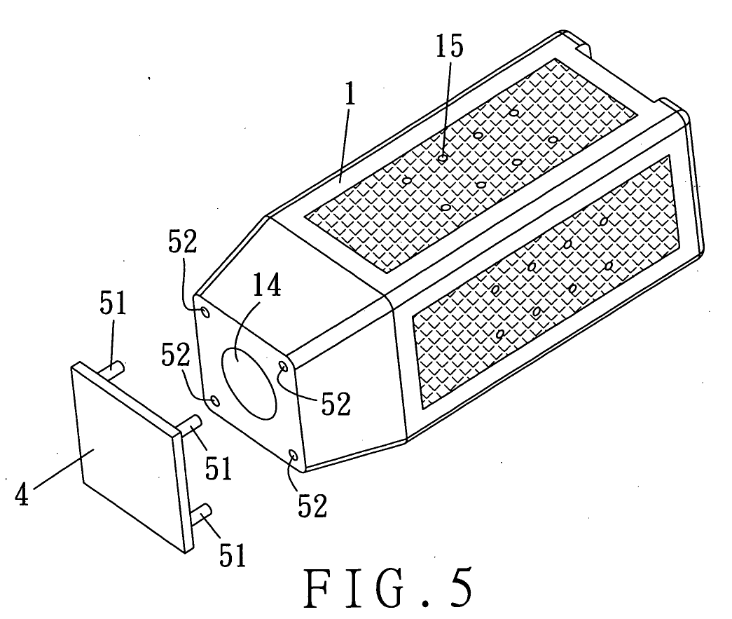

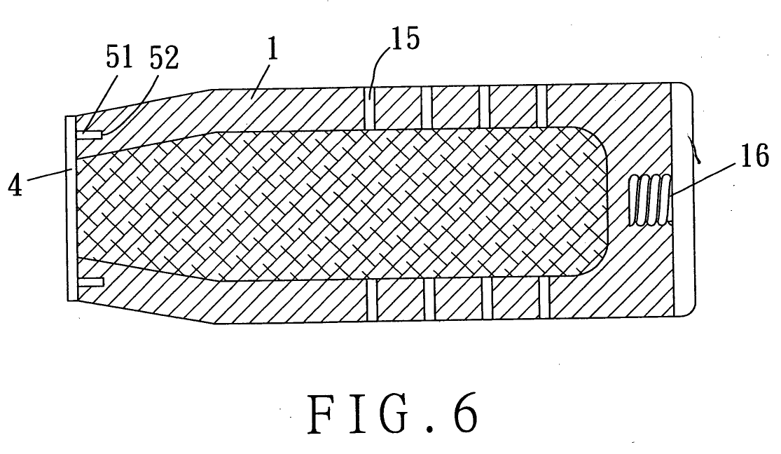

Moreover, FIGS. 5 and 6 show a third preferred embodiment of a body augmenter capable of being inserted with medicine, which has different structures from the second one that an opening sealer 4 is provided with plural male engaging members 51 and the body augmenter 1 is provided with female engaging members 52 so that the male engaging members 51 engage with the female engaging members 52 to close up the opening 14.

While the preferred embodiments of the invention have been described above, it will be recognized and understood that various modifications may be made therein and the appended claims are intended to cover all such modifications that may fall within the spirit and scope of the invention.

What is claimed is:

1. A body augmenter capable of being inserted with medicine, said body augmenter shaped nearly rectangular for easily planted in a vertebra and having a first end and a second end, a cross-sectional area of said first end gradually enlarging to said second end, said body augmenter further having a chamber in its interior for medicine to be stored therein, said chamber extending to a front side surface of said body augmenter and forming an opening for medicine to be inserted therethrough into said chamber, plural holes provided in an outer surface of said body augmenter and communicating with said chamber so that said medicine stored therein may gradually seep therethrough into a human body.

2. The body augmenter as claimed in claim 1, wherein an opening sealer is further provided for sealing up said opening.

3. The body augmenter as claimed in claim 2, wherein said opening sealer is composed of a block of the same size as that of said opening and a plate body that said block is vertically fixed with, with said block fitting tightly in said opening for sealing.

4. The body augmenter as claimed in claim 2, wherein said opening sealer is a plate that is pivotally connected to said body augmenter so that said opening sealer is closed up and opened from said opening by swinging.

5. The body augmenter as claimed in claim 2, wherein said opening sealer is a plate, said opening sealer has plural male engaging members, and said body augmenter has female engaging members, so said male and said female engaging members engage with one another so that said opening sealer is stably combined with said body augmenter.

6. The body augmenter as claimed in claim 1, wherein said second end of said body augmenter is provided with a tool hole for stably supporting a tool therein.
